(19) United States
(12) Patent Application Publication
(10) Pub. No.: US 2007/0183999 A1
(43) Pub. Date: Aug. 9, 2007

(54) Method of preparing a cosmetic composition, and an apparatus for implementing such a method

(75) Inventors: Jean-Francois Grollier, Paris (FR); Roland De La Mettrie, Le Vesinet (FR)

Correspondence Address: OLIFF & BERRIDGE, PLC, P.O. BOX 19928, ALEXANDRIA, VA, 22320, US

(73) Assignee: L'OREAL, PARIS (FR)

(21) Appl. No.: 11/698,176

(22) Filed: Jan. 26, 2007

Related U.S. Application Data
(60) Provisional application No. 60/773,648, filed on Feb. 16, 2006.

(30) Foreign Application Priority Data
Jan. 27, 2006 (FR) 06 50308

(51) Int. Cl. A61K 8/00; A61K 8/18

(52) U.S. Cl. 424/70.1

(57) ABSTRACT

A method of preparing a cosmetic composition includes causing at least one fluid to flow through an enclosure containing at least one substance to be extracted, and collecting the extraction solution in a receptacle. The method further includes delivering more of the same fluid to the receptacle that does not flow through the enclosure so as to obtain a predefined volume of composition in the receptacle.

### CROSS-REFERENCE TO RELATED APPLICATIONS

This non provisional application claims the benefit of French Application No. 06 50308 filed on Jan. 27, 2006 and U.S. Provisional Application No. 60/773,648 filed on Feb. 16, 2006, the entire disclosures of which are herein incorporated by reference.

The present invention relates to cosmetic compositions, and more particularly to those obtained by causing a fluid to flow in contact with a substance having at least one compound that can be extracted by the fluid.

### BACKGROUND

The following European patent applications: EP 1 563 827-A2, EP 1 566 164-A1, EP 1 563 885-A1, EP 1 559 400-A1, EP 1 559 398-A1, EP 1 563 826-A1, EP 1 559 414-A1, EP 1 563 826-A1, EP 1 559 401-A1, EP 1 559 392-A1, EP 1 559 396-A1, and EP 1 556 163-A1, all disclose methods of preparing a cosmetic composition by percolating a fluid.

Application WO 00/56629 discloses a cartridge for an apparatus for extraction by percolation.

### SUMMARY

There exists a need to improve methods of preparing cosmetic compositions.

There also exists a need to prepare personalized cosmetic compositions in a manner that is reliable and relatively simple, and to facilitate the packaging and use of substances involved in preparing such compositions.

Embodiments of the invention seek to satisfy these needs in full or in part.

Thus, in one of its aspects, embodiments of the invention provide a method of preparing a cosmetic composition, the method comprising:

a) causing at least one fluid to flow through at least one enclosure containing at least one substance to be extracted, and collecting at least one extraction solution in a receptacle; and

b) delivering more of the same fluid to the receptacle without causing it to flow through the enclosure.

Steps a) and b) are performed so as to obtain a predefined volume of composition in the receptacle.

The two steps a) and b) may be successive, in the order a) then b) or vice versa, or they may be simultaneous at least in part.

By way of example, embodiments of the invention make it possible to obtain a composition having a desired concentration of at least one compound by diluting, to a greater or lesser extent, a solution that has been extracted by the fluid.

If so desired, embodiments of the invention also make it possible to obtain a predefined quantity of composition regardless of the quantity of fluid used for extraction purposes.

In embodiments, a plurality of compositions can be prepared from at least one enclosure, the compositions having different characteristics depending on the degree of dilution desired, for example, thereby making it possible to personalize the composition.

The enclosure may be a compartment of a refill, said refill possibly being in the form of a cartridge.

Embodiments of the invention can also make it easier to package and store substance(s) used for preparing the composition, by making it possible to use substances that are not liquids, for example.

Embodiments of the invention also make it possible to have available within a common enclosure compounds in powder form that are unsuitable for being stored over a long period when in solution, in particular when mixed together.

Finally, embodiments of the invention can make it easier to use substances of natural origin, in particular plant extracts or minerals.

The flow of fluid through the enclosure may be by percolation. The term “percolation” designates passing a fluid through a medium that is permeable to the fluid, the medium being in powder form, for example. The medium may be dissolved entirely by the passage of the fluid, where appropriate. The medium need not be a foodstuff.

The receptacle, e.g. a bowl, may optionally be provided with a closure member, in particular for stirring purposes or for enabling the consumer to transport the composition for use other than at the preparation site.

The receptacle may optionally contain at least one compound for mixing with the solution(s) obtained by extraction, in particular a cosmetically acceptable medium, e.g. a dye oxidizer or a base.

The fluid, which is sometimes also known as a percolate, may comprise a liquid and/or a gas, e.g. water. Other solvents can be used, e.g. a solution in alcohol or in oil.

The quantity of substance contained in the enclosure in which injection takes place may be relatively small, e.g. corresponding to a single use. The quantity of substance may be less than or equal to 25 cubic centimeters (cm³), or 10 cm³, or 7.5 cm³, or indeed 5 cm³, for example.

Once extraction has been performed by the fluid, the enclosure can be disposed of, e.g. by being recycled.

The quantity of fluid that is delivered in step a) and/or in step b) may be determined by weighing the receptacle and its content, or by measuring the volume of substance contained in the receptacle. The quantity of fluid that is injected into the enclosure may be determined as a function of at least one characteristic of the composition, e.g. the color of said composition or its dyeing capacity. The quantity of fluid that is delivered to the receptacle without flowing through the enclosure may seek to obtain a constant volume for the composition and/or it may be determined as a function of at least one characteristic of the composition that is to be obtained.

Prior to selecting the quantity of fluid, whether it be the quantity for injecting into the enclosure or the quantity for delivering directly to the receptacle, at least one item of data may be acquired that is associated with the person to be treated with the composition, and the quantity of fluid may be selected on the basis of said at least one item.

A plurality of enclosures may be disposed on the extractor apparatus, thus making it possible to make a mixture, for example. The enclosures may optionally belong to a single refill.

The fluid may flow simultaneously or otherwise through the various enclosures.

The fluid may be injected into a plurality of enclosures with the help of a plurality of injectors, which may be associated respectively with different enclosures. The use of a plurality of injectors can reduce the risk of an enclosure becoming contaminated by a substance coming from another enclosure. Where appropriate, the injector(s) may be subjected, between the fluid injection steps, to cleaning, e.g. using a flow of fluid in and/or over the injector. The injector(s) may also be removable in order to make it/them easier to clean.

At least one of the enclosures may include an agent for coloring keratinous materials, e.g. the skin, mucous membranes, hair, and other keratinous fibers such as the eyelashes and the eyebrows.

Different enclosures in a single refill may contain different coloring agents, or the same coloring agents but at different concentrations.

The same refill may include coloring agents that enable a plurality of shades of a given color to be obtained.

The coloring agent(s) may be for performing dyeing directly, or by oxidation, optionally for bleaching purposes. For example, it may constitute tone on tone coloring.

The coloring agent(s) may be direct dyes or oxidation dyes.

When there are a plurality of enclosures, prior to selecting at least one enclosure in which injection is to be performed, at least one item of data may be acquired that is associated with the person to be treated with the cosmetic composition or that is associated with a characteristic to be obtained, and the enclosure(s) in which injection takes place may be selected on the basis of said at least one item. The data may comprise a color that is to be reproduced, e.g. that of the skin or the hair of the person to be treated with the composition.

By way of example, selection may also be for the purpose of reproducing a color selected by a user from a color chart or a hair color chart or corresponding to a commercial reference.

The substances contained in the enclosures may be in powder form. Where appropriate, a single enclosure may contain at least two compounds in powder form. These two compounds may be mixed, optionally uniformly, within the enclosure. By way of example, the compounds may be placed in the enclosure in successive layers.

The temperature of the fluid injected into the enclosure may be greater than 80° C., thus encouraging certain compounds to dissolve, the fluid possibly being in the form of both vapor and liquid phases during injection, thus facilitating extraction.

The injection pressure is preferably greater than or equal to 1 bar (10⁵ Pa), or greater than or equal to 3 bars, or more preferably greater than or equal to 10 bars, e.g. being as high as 10 bars to 50 bars.

The enclosure(s) may be defined at least in part by a first wall suitable for having an injector of the extractor apparatus pass therethrough, e.g. by being perforated.

The enclosure(s) may also be defined at least in part by a second wall suitable for opening under the effect of the pressure at which the fluid is injected into the enclosure, e.g. by the second wall being deformed against at least one portion in relief that is suitable for causing it to open. By way of example, the second wall is deformed against a grid arranged so that deformation is accompanied by the enclosure opening, while also serving to enable the second wall to retain substantially all of the substance that has not been dissolved by the fluid flowing through the enclosure.

By way of example, the first and second walls comprise at least one layer of material that is suitable for being tom and/or perforated relatively easily, e.g., a layer comprising a metal, e.g. aluminum, of thickness less than or equal to 0.1 millimeters (mm), for example.

In another of its aspects, embodiments of the invention also provide an apparatus for preparing a cosmetic composition by causing a fluid to flow through at least one enclosure containing at least one substance to be extracted, the apparatus comprising:

a support system for supporting the enclosure; and

an injector system for:
firstly injecting the fluid through the enclosure so as to produce at least one solution that is collected in a receptacle; and
secondly delivering more of the same fluid to the receptacle without the fluid flowing through the enclosure.

The support system may be adapted to receive a plurality of enclosures.

The extractor apparatus may be arranged in such a manner as to make it possible to select one or more enclosures.

At least one of the injector system and the support system may be arranged in such a manner as to make it possible to select the enclosure(s) in which the fluid is to be injected.

The enclosures may be fitted on the extractor apparatus or they may belong thereto.

The enclosures may optionally belong to a single refill. The refill may comprise a cartridge containing a plurality of enclosures. The refill may also comprise a plurality of capsules that are secured to one another, at least during use, defining respective different enclosures.

The apparatus may include a system for collecting solutions that come from fluid flowing through the enclosures, which system may be arranged to mix the solutions.

The collector system may comprise a receptacle placed under the enclosures and/or channels enabling the flow of liquid to be guided after it has passed through the enclosures.

The apparatus may comprise a plurality of injectors.

Each enclosure may be associated with at least one sealing gasket arranged to co-operate with at least one injector. By way of example, the sealing gasket may be a membrane of an elastically deformable material that is arranged to allow the injector to pass therethrough.

Each enclosure may be substantially airtight.

When there are a plurality of enclosures, the support system may be arranged to enable the enclosures to be moved relative to the injector system. This movement may be performed manually or it may be motor-driven, it may be in rotation, or in translation, or in some other manner, e.g. in such a manner as to cause at least one injector to penetrate into a selected enclosure. During such movement, the injector may follow a trajectory during which it passes in register with at least one other enclosure, for example.

The extractor apparatus may also include a plurality of injectors, and optionally that can avoid any need for such movement.

Selecting the enclosure(s) in which injection is to be performed may also be accomplished by enabling the user to position the refill in the extractor apparatus in such a manner that injection takes place in one or more selected enclosures.

Where appropriate, the user may select only one enclosure at a time, perform injection, and reposition the refill in the extractor apparatus so as to select another enclosure, in the event that the composition is to be prepared from substances coming from a plurality of enclosures.

As mentioned above, the extractor apparatus may include a heater system to raise the temperature of the fluid to greater than 80° C. prior to injection.

When the enclosure(s) include at least one coloring agent, the extractor apparatus may include a selector system arranged firstly to enable a user to select one tint from a plurality of tints, and secondly to confirm injection of the fluid so as to collect a cosmetic composition of tint that corresponds to the selected tint. By way of example, the selector system may act on the quantity of fluid that is delivered without passing through the enclosure, and it can optionally act on the quantities of fluid that are delivered to the enclosure(s).

The extractor apparatus may include a sensor making it possible to determine the quantity of composition present in the receptacle. By way of example, the sensor is a weight and/or volume sensor making it possible to evaluate the quantity of composition present in the receptacle.

In embodiments of the invention, each time a liquid has been delivered to the receptacle, the weight and/or the volume of substance present in the receptacle is determined substantially in real time by weighing said receptacle and/or by measuring the volume of composition contained in said receptacle, and dispensing of the fluid can be stopped once the intended quantity has been dispensed.

The extractor apparatus may also include a level sensor for determining the level of liquid in the receptacle and/or in a reservoir containing the fluid to be injected, for example.

The extractor apparatus may include at least one control member making it possible to modify the duration of fluid injection into the enclosure, for example. The extractor apparatus can be arranged to deliver the quantity of additional fluid directly to the receptacle, regardless of the above-mentioned duration of injection, making it possible to obtain a predefined volume of composition, the volume optionally being adjustable by the user. The control member may include a rotary or slider control knob that is optionally associated with graduations.

The extractor apparatus may be arranged to receive at least one item of data that relates to the concentration of at least one substance in the solution collected in the receptacle as a function of the duration of injection into the enclosure containing the substance, and it can be arranged to calculate the quantities of fluid to be injected into the enclosure and into the receptacle as a function of said item so as to obtain a desired concentration in the receptacle.

By way of example, the item of data is generated by a computer system from an identifier present on the refill.

### BRIEF DESCRIPTION OF THE DRAWINGS

Various details of the present invention may be better understood by reading the following detailed description of non-limiting embodiments, and on examining the accompanying drawings, in which:

FIG. 1 is a diagrammatic perspective view of an example of a refill containing a cosmetic substance to be extracted;

FIG. 2 is a cross-sectional view of the FIG. 1 refill;

FIG. 3 is a diagrammatic and fragmentary perspective view showing an example of an extractor apparatus;

FIG. 4 is a simplified diagram of an example of a feed circuit for the extractor apparatus;

FIG. 5 is a view similar to FIG. 2 showing the refill in place in the extractor apparatus;

FIG. 6 shows an embodiment of the invention being implemented with a plurality of enclosures;

FIG. 7 is a view similar to FIG. 4 showing a variant embodiment of the feed circuit;

FIG. 8 is a view similar to FIG. 1 showing a variant embodiment of the refill;

FIG. 9 is a cross-sectional view of the body of the FIG. 8 refill;

FIG. 10 is a view similar to FIG. 2 showing a variant embodiment of the refill;

FIG. 11 is a view similar to FIG. 3 of a variant embodiment of the extractor apparatus;

FIG. 12 is a diagrammatic perspective view of another example of a refill and the corresponding positioning of the injectors;

FIG. 13 is a cross-sectional view taken along XIII-XIII of FIG. 12;

FIG. 14 is a plan view of another embodiment of the refill;

FIG. 15 is a cross-sectional view taken along XV-XV of FIG. 14;

FIG. 16 is a plan view of a variant embodiment of the refill; and

FIG. 17 is a fragmentary and diagrammatic view of a variant embodiment of the extractor apparatus while using the FIG. 16 refill.

### DETAILED DESCRIPTION OF EMBODIMENTS

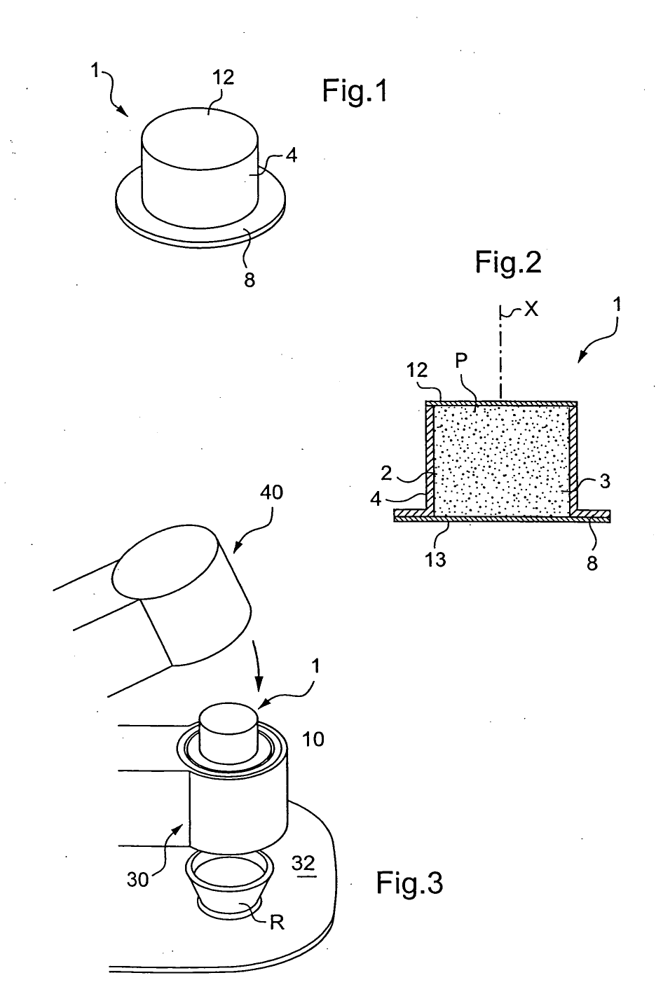

FIGS. 1 and 2 show a refill 1 containing at least one cosmetic substance P. The word “refill” is used because a user could refill (replenish) the user's supply of the substance P by purchasing one or more refills 1 when desired (for example, when the user's supply of substance P is depleted). The refills 1 are not themselves necessarily refilled with substance P (that is, they are not necessarily re-used), although in embodiments they could be refilled with substance P.

The refill 1 comprises a tubular body 4 and first and second walls 12 and 13 defining an enclosure 2 containing the substance P.

In the example shown, the first wall 12 is made as a single piece together with the body 4, e.g. by molding plastics material or by stamping sheet material including a metal layer, for example.

By way of example, the second wall 13 is a film that can include at least one layer of a metal, and that is fastened, e.g. heat sealed, on a flange 8 that is made integrally with the body 4.

In order to manufacture the refill 1, the person skilled in the art can easily refer to refills that exist for preparing beverages of the “espresso” type by percolation.

By way of example, the substance P is powder and can be extracted by causing a fluid to flow through the enclosure 2, the concentration of substance in the solution leaving the enclosure being a function of the duration of injection and of the conditions of injection, for example.

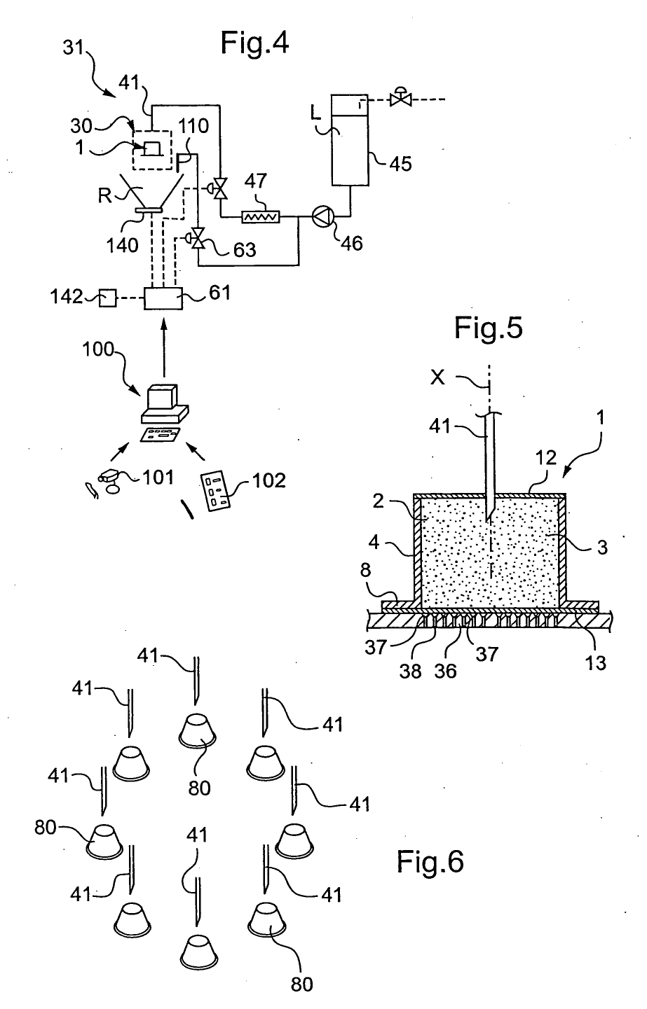

In order to use the refill 1 of FIG. 1, the user may place it on a support system 30 of an extractor apparatus 31 that also includes an injector system 40, as shown in FIGS. 3 and 4.

In the example shown, the extractor apparatus 31 includes a gap 32 under the support system 30 for receiving a receptacle R suitable for collecting a solution coming from percolation of a fluid through the enclosure 2.

As shown in FIG. 5, the support system 30 can include a grid 36 provided with portions in relief 37 against which the second wall 13 can be pressed under the pressure of the injected fluid.

The second wall 13 is arranged to be perforated, during injection, by the portions in relief 37 of the grid, under the effect of the pressure existing in the enclosure, thereby enabling the injected fluid to flow through the channels 38 in the grid 36 towards the receptacle R.

The second wall 13 may advantageously be opened in such a manner as substantially to retain inside the enclosure in question any particles of the substance P that have not dissolved.

The receptacle R may be wide enough to collect the solution coming from the enclosure, given its position. Where appropriate, a collector system may be provided to convey the solution into the receptacle when the enclosure is not situated completely vertically thereover.

The injector system 40 includes at least one injector 41 capable of passing through the first wall 12 and of penetrating into the selected enclosure.

The extractor apparatus 31 may include sealing means (not shown) making it possible, where necessary, to guarantee that the injector 41 penetrates in leaktight manner into the selected enclosure.

By way of example, these sealing means may comprise at least one sealing gasket that bears against the outside of the refill, at least during injection.

As shown diagrammatically in FIG. 4, the extractor apparatus 31 may include an extractor fluid feed circuit having: at least one tank 45 for containing the liquid L that is to be injected; a pump 46; and a heater member 47 serving to raise the liquid L to the desired temperature, and where appropriate to generate steam. This can contribute to generating the desired injection pressure.

In the example described, the fluid injection pressure is, for example, at least 3 bars (3×10⁵ Pa) and preferably at least 10 bars, and the injection temperature is at least 80° C., for example.

In particular, the temperature of the fluid may be sufficient for the fluid to present both liquid and gaseous phases while it is being injected into the enclosure.

The tank 45 of the extractor apparatus may be filled manually or automatically, being connected to a water pipe, e.g. via a solenoid valve.

Where appropriate, the tank 45 may be removable so as to make it easier to fill manually.

The heater member 47 may comprise an electrical resistor element.

The extractor apparatus 31 may also include a level sensor that is useful for detecting when it is necessary to refill the tank 45 or to enable the receptacle R to be filled with a predefined total quantity of composition, for example.

The extractor apparatus includes an output 110 enabling the liquid L to be dispensed into the receptacle R without contact with the substance(s) contained in the refill, e.g. for the purpose of diluting the solution(s) obtained by percolation into the receptacle so as to obtain a predefined volume and/or concentration of composition.

The output 110 may be fed using a solenoid valve 63 or any other means for controlling the quantity of fluid that is dispensed, such as a pump, for example.

The solenoid valve 63 can be controlled by a control system 61, e.g. so as to have a final volume of composition in the receptacle that corresponds to a predefined value.

The extractor apparatus can include a weight sensor 140 that is placed under the receptacle R, and that is useful for determining the quantity of fluid that has been delivered to said receptacle.

The weight sensor 140 can be connected to the control system 61.

The extractor apparatus can include a control member 142 making it possible to modify the duration of fluid injection into the enclosure, so as to obtain a greater or lesser concentration of extracted substance, for example.

The control system 61 can be arranged to control the operation of the feed circuit so as to obtain, regardless of the setting of the control member, a constant volume of composition in the receptacle by means of the data that is delivered by the weight sensor 140, for example.

By way of example, the control member 142 includes a rotary or slider knob, and the extractor apparatus can include graduations enabling the user to locate a particular setting.

FIG. 4 also shows the possibility of the feed circuit exchanging data with a computer system 100, e.g. comprising at least one microcomputer, a personal digital assistant (PDA), a terminal connected to a network, or a mobile telephone, or even that is integrated in the extractor apparatus.

By way of example, the computer system 100 may be associated with a data acquisition system, e.g. comprising a camera 101 or any other device for analyzing keratinous materials, e.g. for analyzing the color of the skin or of the hair.

The computer system 100 may also be associated with a color chart or with a hair color chart 102 or with some other evaluation tool, e.g. a questionnaire.

The computer system 100 may be arranged in such a manner as to enable a user to select at least one characteristic of the cosmetic composition that is to be prepared. By way of example, this characteristic may be color.

The computer system 100 may be used to enable the user to select a desired tint, and the computer system 100 may be arranged to send data to the control system 61 enabling it to control the injection of fluid in such a manner as to obtain a composition having the desired properties.

For example, the enclosure 2 can contain at least one coloring agent and the control system 61 can control the quantities of fluid that are injected into the enclosure 2 and into the receptacle R without passing through the enclosure, so as to obtain a composition having the desired concentration of coloring agent.

By way of example, the control system 61 can be arranged so that, regardless of the tint requested from amongst the tints that can be obtained, the same volume of composition is prepared in the receptacle. This can make it easier to apply the composition and/or mix it with a compound that is initially present in the receptacle, or that is added to the composition, e.g. a dye oxidizer or a base.

In the presence of a computer system 100, the extractor apparatus need not include a control member 142 as described above, the operation of the feed circuit depending only on signals received by the control system 61 and coming from the computer system 100, for example.

Where appropriate, the person skilled in the art can easily refer to the extractor apparatuses used for preparing beverages of the “espresso” type, and the feed circuits thereof can be reproduced, at least in part.

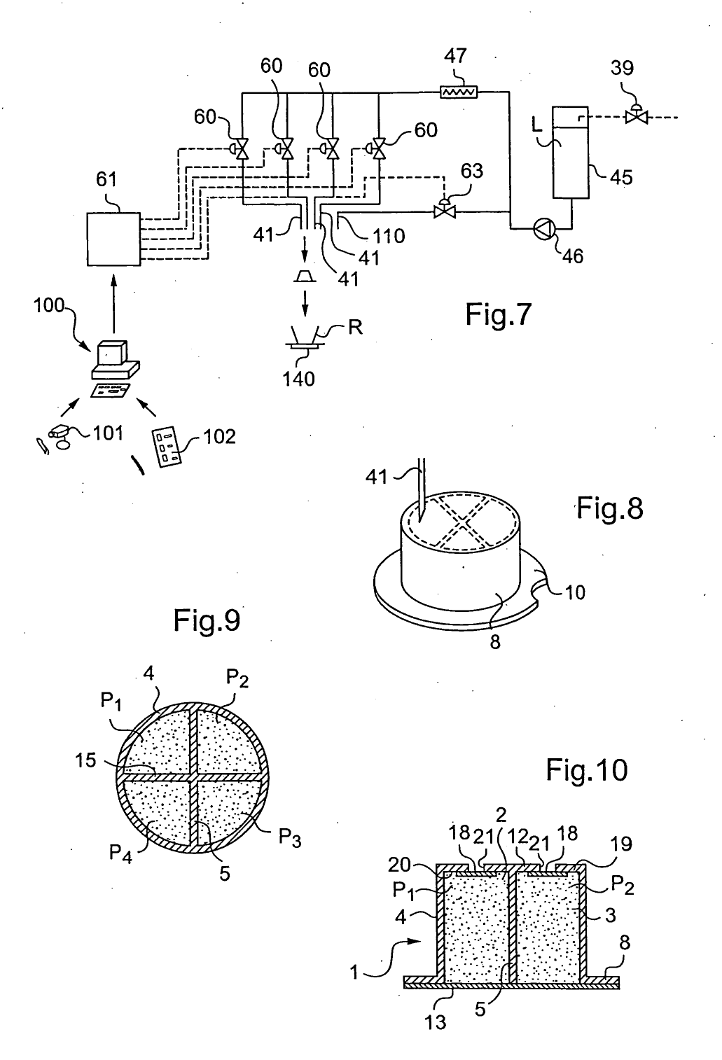

By way of example, such apparatuses are disclosed in the following publications: AT 168 405, U.S. Pat No. 2,688,911, DE 3 243 870, IT 1 265 636, and WO 2004/006 740-A2.

Where appropriate, and as shown in FIGS. 6 and 7, the extractor apparatus can include a plurality of injectors 41 that are respectively associated with a plurality of enclosures that are defined by independent capsules 80, for example. The enclosures can contain different substances. The support system of the extractor apparatus includes a variety of housings for receiving the capsules, for example.

Injection may be performed into a selected capsule by causing the injector to penetrate into the corresponding enclosure, e.g. by moving the injector and/or the capsule, where this movement may be caused, the example, by the user acting on at least one of the injector system and the support system. This movement may also be the result of drive from a pneumatic or a hydraulic motor, e.g. with assistance from the pressure of the fluid.

The feed circuit may have a plurality of flow-control members 60, each associated with a respective injector 41 and controlled in common by a control system 61 in such a manner that only the selected injector(s) 41 is/are fed by the fluid for injection.

The quantity of fluid injected into the selected enclosure(s) may be predefined, or, for example, selected as a function of the concentration desired for the or each compound extracted by percolation.

By way of example, the flow-control members 60 may be proportional solenoid valves enabling the flow through each injector to be adjusted in a manner other than by on/off adjustment alone. For example, the control members 60 may be piezoelectric.

By adjusting the different flow rates several shades can be obtained, for example.

By way of example, the injectors 41 are arranged in such a manner that all of them penetrate into the enclosures, with fluid being injected into only the selected enclosure(s) by controlling the flow.

In a variant, the injector system is arranged in such a manner that the injectors and/or the corresponding enclosures can be moved relative to one another so that only the injector(s) that is/are being fed penetrate(s) into the corresponding enclosure(s). By way of example, the pressure of the injection fluid can contribute to moving injector while it is being fed, so as to cause it to penetrate into the enclosure.

A plurality of enclosures can also be associated with a single refill.

By way of illustration, FIGS. 8 and 9 show a refill 1 in the form of a cartridge.

The refill 1 contains several substances P₁ to P₄ respectively contained in separate manner in four enclosures defined by the refill.

In the example shown, the enclosures are formed by compartments of the body 4 that are separated by partitions 5 that are made by being molded as a single piece together with the body 4, for example.

The flange 8 may be provided with keying means 10, e.g. in the form of a recessed or projecting portion in relief, e.g. a notch.

Alternatively, if the body 4 is made without keying means 10, it could be made to be of a shape that is not circularly symmetrical, so as to allow it to be positioned in the associated extractor apparatus in one way only.

The volume of each of the enclosures is preferably less than or equal to 25 cm³ or 10 cm³, e.g. lying in the range 1 cm³ to 5 cm³.

In the variant in FIG. 10, the refill 1 may have a first wall 12 which is pierced by openings 21 associated respectively with the different enclosures. The openings 21 may be closed in such a manner as to enable the injectors of the associated extractor apparatus to enter therethrough. By way of example, closing may be performed using caps 18 heat sealed to the inside face 20 of the first wall 12. In a variant, the caps 18 are secured to the outside on the first wall 12 or they are injected thereover, e.g. using an elastomer material.

The caps 18 may also be made integrally with the first wall 12 and out of the same material, by giving the first wall 12 reduced thickness locally, or by making zones of weakness. While injection is taking place, the caps 18 may be arranged to be perforated or to become detached, at least in part, from the first wall 12.

By way of example, the refill 1 contains one or more agents for coloring keratinous materials, in different colors.

The user can select the enclosure containing the substance corresponding to the desired color.

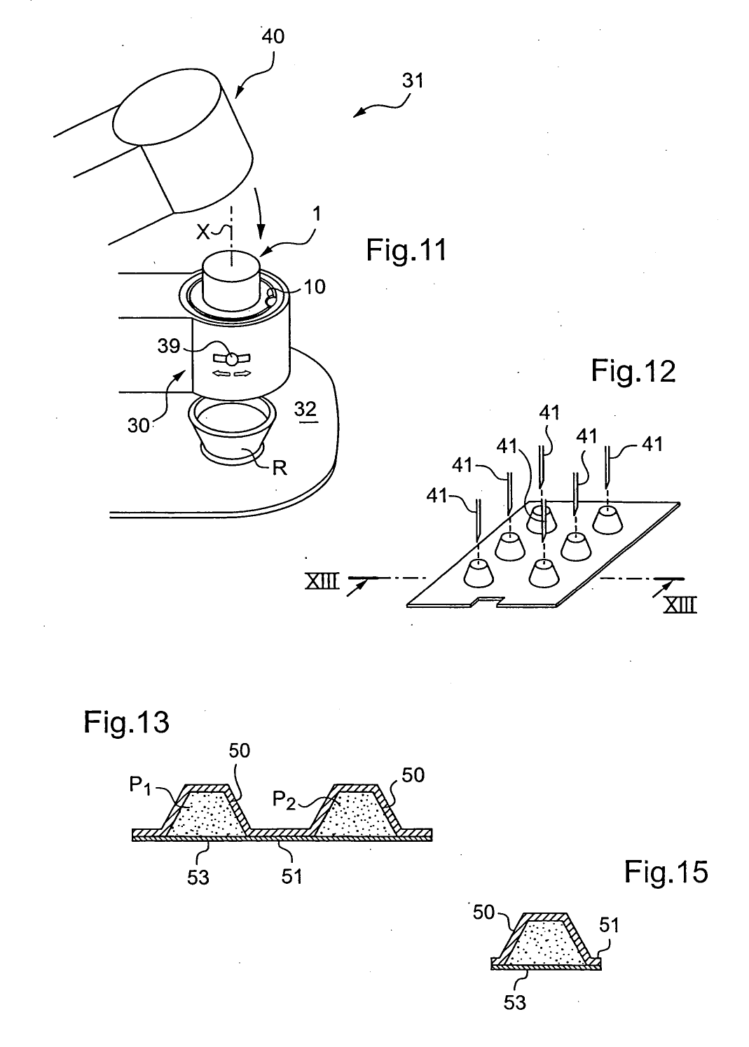

When the extractor apparatus includes a single injector only, in order to enable the user to select the enclosure into which the fluid is injected, the extractor apparatus 31 may, as shown in FIG. 11, include a selector system, e.g. comprising a control knob 39 which the user can actuate in order to turn the refill 1 about it axis X so as to position it with an angular orientation that enables the injector 41 to penetrate into the enclosure in which percolation is to be performed.

If necessary, the user can perform several injections in succession in different enclosures and can collect the solutions that result from percolation in a single receptacle, in order to mix them.

For example, the user may select at least two enclosures enabling a particular color shade or coloring agent to be obtained by mixing the extraction solutions.

The refill 1 may be in a form other than that of a cartridge, and for example as shown in FIGS. 12 and 13, it may have enclosures defined by capsules 50 carried by a support element 51, e.g. in the form of a plate.

By way of example, the capsules 50 may have a first wall that is formed by stamping or thermoforming a sheet material, e.g. aluminum or a laminate including aluminum. As stamped or thermoformed, this wall may define the support element 51 around the capsules 50.

The capsules 50 may be closed by a second wall 53, e.g. constituted by a film that is heat sealed on the support element 51.

The support element 51 may include keying means 56, e.g. the form of a notch.

Where appropriate, the support element 51 and the capsules 50 may be made separately.

For example, the support element 51 can be made in the form of a plate with holes, and the capsules can be secured in the holes, e.g. as a function of the substances that it is desired to associate within a single refill.

The capsules may be fastened in the holes of the support element in optionally releasable manner. Where appropriate, releasable fastening can enable the user to replace used capsules with new capsules. By way of example the releasable fastening may be performed by friction and/or by snap-fastening.

In the example of FIGS. 12 and 13, the capsules 50 are disposed in rows.

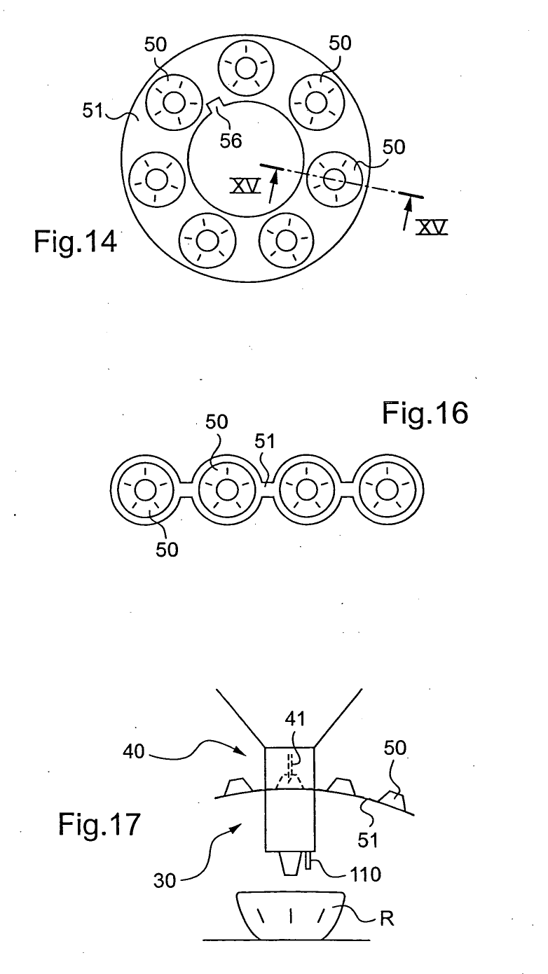

In the variant of FIGS. 14 and 15, the support element 51 is annular in shape, and in the example of FIG. 16, it is in the form of a strip.

In order to use the refill of FIG. 16, the user can place it, for example, in an extractor apparatus between a support system 30 and an injector system 40, as shown in FIG. 17.

The user positions the support element 51 in the extractor apparatus as a function of the enclosure into which it is desired to inject fluid. Only the capsule corresponding to said enclosure is perforated by the injector 41.

Refills including a plurality of enclosures can also be used with extractor apparatuses including a plurality of injectors.

When injection takes place into a plurality of enclosures, the quantity of fluid that is delivered directly to the receptacle can be determined so as to obtain a predefined final volume of composition.

The invention is not limited to the embodiments shown.

In particular, the refill may be given further shapes other than those shown, the example in the form of a small tray, a sachet, etc. Capsules or sachets containing different substances may be contained in a single package.

The enclosures may be filled at the time of manufacture. In a variant, the enclosures may be filled as a function of requests made by a consumer, for example.

Different compartments in a single refill may be made separately and then assembled together.

During manufacture, the enclosures may also be in a deployed configuration, and this configuration can be modified during manufacture and/or in use.

Fluid may flow in a selected enclosure either vertically or otherwise.

In the examples shown, injection into a selected enclosure takes place via a single injector, however in a variant injection may take place via a plurality of injectors, in order to improve putting the fluid into contact with the substance contained in the enclosure.

A refill defining one or more enclosures can be associated with at least one item of data enabling the extractor apparatus or a computer system associated with the extractor system to track variation in the concentration of extracted substance as a function of the quantity of fluid injected and of injection conditions, where appropriate.

This can make it easier to calculate the quantities of fluid that are to be delivered to the enclosure and to the receptacle in order to obtain a desired concentration.

By way of example, the data associated with the refill includes an identifier.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention.

The term “comprising a” should be understood as being synonymous with “comprising at least one”, unless specified to the contrary.

What is claimed is:

1. A method of preparing a cosmetic composition, the method comprising:
a) causing at least one fluid to flow through at least one enclosure containing at least one substance to be extracted, and collecting at least one extraction solution in a receptacle; and
b) delivering more of the same fluid to the receptacle without causing it to flow through the enclosure so as to obtain a predefined volume of composition in the receptacle.

2. A method according to claim 1, with step b) following step a).

3. A method according to claim 1, in which a quantity of fluid that is injected into the enclosure is determined as a function of at least one characteristic of the composition that is to be obtained.

4. A method according to claim 1, in which a quantity of fluid that is to be delivered to the receptacle without flowing through the enclosure is determined as a function of at least one characteristic of the composition that is to be obtained.

5. A method according to claim 3, the characteristic including a color or a dyeing capacity of the composition.

6. A method according to claim 3, in which at least one item of data is acquired that is associated with a person to be treated with the composition, and in which the quantity of fluid is determined on the basis of said at least one item of data.

7. A method according to claim 1, in which a quantity of fluid that is to be delivered in step b) is selected so as to have a constant volume of composition in the receptacle regardless of the quantity that is delivered in step a).

8. A method according to claim 1, in which a quantity of fluid that is delivered in step a) and/or in step b) is determined by weighing the receptacle and its content.

9. A method according to claim 1, in which a quantity of fluid that is delivered in step a) and/or in step b) is determined by measuring the volume of substance contained in the receptacle.

10. A method according to claim 1, the fluid including water.

11. A method according to claim 10, the fluid being water.

12. A method according to claim 1, the fluid being injected into the enclosure at a temperature greater than 80° C.

13. A method according to claim 1, the fluid being injected into the enclosure under a pressure greater than or equal to 1 bar.

14. A method according to claim 13, wherein the pressure is greater than or equal to 3 bars.

15. A method according to claim 14, wherein the pressure is greater than or equal to 10 bars.

16. A method according to claim 1, the fluid injected into the enclosure being in a form of both vapor and liquid phases.

17. A method according to claim 1, the fluid that is delivered to the receptacle without flowing through the enclosure being at a temperature less than or equal to 80° C.

18. A method according to claim 1, the enclosure being selected from a plurality of enclosures containing different substances and being disposed on a common extractor apparatus and/or being contained in a single body.

19. A method according to claim 18, the enclosures being contained in the single body.

20. A method according to claim 18, the enclosures not being contained in a single body.

21. A method according to claim 1, in which the enclosure(s) are defined at least in part by a first wall suitable for being pierced by an injector of an extractor apparatus, and by a second wall capable of opening under the effect of the of the pressure of fluid being injected into the enclosure.

22. An apparatus for preparing a cosmetic composition by causing at least one fluid to flow through at least one enclosure containing at least one substance to be extracted, the apparatus comprising:
a support system for supporting the enclosure; and
an injector system for:
firstly injecting the fluid through the enclosure so as to produce at least one solution that is collected in a receptacle; and
secondly delivering more of the same fluid to the receptacle without the fluid flowing through the enclosure.

23. An apparatus according to claim 22, the support system being arranged to receive a plurality of enclosures.

24. An apparatus according to claim 22, including a weight and/or volume sensor arranged to evaluate the quantity of substance in the receptacle.

25. An apparatus according to claim 22, including a control system arranged to control the quantity of fluid that is delivered to the receptacle without contact with the substance(s) contained in the enclosure.

26. An apparatus according to claim 22, the injector system injecting the fluid under a pressure of at least 3 bars and at a temperature greater than 80° C.

27. An apparatus according to claim 26, the injector system injecting the fluid in a form of both vapor and liquid phases.

28. An apparatus according to claim 22, the injector system injecting water as the fluid.

29. An assembly comprising:
an apparatus as defined in claim 22; and
at least one enclosure containing at least one cosmetic substance that is to be extracted by causing a fluid to flow through the enclosure.
